(19) United States
(12) Patent Application Publication
(10) Pub. No.: US 2019/0083027 A1
(43) Pub. Date: Mar. 21, 2019

(54) SPINAL ASSESSMENT SYSTEM

(71) Applicant: The Governors of the University of Alberta, Edmonton (CA)

(72) Inventor: Greg Kawchuk, Edmonton (CA)

(73) Assignee: The Governors of the University of Alberta, Edmonton, AB (CA)

(21) Appl. No.: 16/088,002

(22) PCT Filed: Mar. 29, 2017
(86) PCT No.: PCT/CA2017/050386

§ 371 Date: Sep. 24, 2018

Related U.S. Application Data
(60) Provisional application No. 62/314,522, filed on Mar. 29, 2016.

(51) Int. Cl. A61B 5/00; A61B 5/107; A61B 5/11

(52) U.S. Cl. CPC A61B 5/4566; A61B 5/1116; A61B 5/1079

(57) ABSTRACT

A device for measuring spinal stiffness comprises a frame-work supporting a roller that is rolled over the surface of the back. The device creates and records a trajectory across a subject, using a control board and the roller, and moves the roller along the trajectory while applying a force to the subject using the roller and records displacement of the object in the direction of the force. A stiffness map is created using displacement as a function of force. Motors may be used to move the roller through three axes.

### TECHNICAL FIELD

Diagnostic medical system

### BACKGROUND

It is well-known that tissue stiffness changes with pathology. For example, glaucoma causes an increase in the stiffness of the eye. Historically, clinicians have monitored tissue stiffness for pathological change with palpation. In glaucoma for example, clinicians would ask a patient to shut their eyes then gently push on the eyeball to see if it felt more stiff than usual. Unfortunately, palpation has been shown to have limited value in detecting small changes in stiffness that are the first indicators of a change in tissue status. Fortunately, palpation of the eye has been replaced by technologies able to measure stiffness non-invasively with increased sensitivity, reliability, accuracy and safety.

A similar situation exists for low back pain, the most common and costly musculoskeletal disability in the world. In back pain, pathological change to the tissues, injury and degeneration all alter the stiffness of the spine. Unlike glaucoma, palpation remains the current standard for assessing spine stiffness no matter the discipline of the clinician (e.g. physical therapist, physician, chiropractor) or the intended intervention (e.g. manipulation, surgery).

In prior art, the measurement of the wheel in the vertical direction is made by a ruler printed on the rod. This would be an inaccurate way of taking this measure and would not result in the level of resolution that we now know is required to measure changes in stiffness as a result of treatment. This prior form of measurement is also made less accurate in that there is no mention of breathing control. If the subject were breathing in/out during the measure, then the measure would continuously change. Second, the vertical measure is taken by “eye” which is problematic for consistency. In other words, the operator must look at the ruler and then eyeball what they think they see on the ruler and then writes it down. These problems are solved in the new device by using an electronic sensor to measure vertical rod displacement to hundredths or thousandths or a millimeter and record those measurements automatically.

Traditionally, devices could not move in all directions within the horizontal plane without repositioning the subject underneath the device, and so it was not possible to assess the spine sufficiently. Clinicians, however, require stiffness data that comes from the spine and most spines are not straight, especially those requiring clinical assessment. U.S. Pat. No. 5,101,835 discloses movement of the roller in the head-toe direction in the prior art, but there is no measurement of this movement. What is described is that by hand, the operator takes measurements at specific points then links those points together in a hand drawing. The result would be extremely inaccurate as the operator has to interpret how the curve is drawn between the data collection points.

The inventor has developed an alternative to the practice of using palpation to assess stiffness; a mechanized probe to measure spinal stiffness with high levels of reliability, accuracy, sensitivity, as shown in WO2009140756 published Nov. 26, 2009. The inventor has shown that spinal stiffness changes with pathology and can be returned to normal values with treatment. As such, spinal stiffness measurement show significant clinical promise as it is one of only a handful of objective measurements related to back pain status. This probe is somewhat expensive, and requires a lengthy analysis with possibly two operators.

### SUMMARY

A method is disclosed comprising creating and recording a trajectory across a subject; using a control board and an object supported by a framework and while applying a force along a direction z to the subject using the object, moving the object along the trajectory and recording displacement of the object in the direction z; and using displacement as a function of force to determine a stiffness map corresponding to the trajectory.

In various embodiments, there may be included any one or more of the following features: creating a trajectory comprises moving a light beam across the subject and recording x and y coordinates of the light beam; moving the object comprises operating motors on the framework; creating a trajectory comprises moving the object and recording movement of the object using sensors; the object comprises a roller; recording displacement is carried out iteratively for different forces; the trajectory is across a non-spine portion of the back of the subject; the trajectory is across the spine of the subject; the stiffness map is associated with a pain record; the method is carried out only while the subject is holding breathing.

An apparatus comprises a control board; a framework providing controlled movement of an object in x, y and z dimensions, the control board being connected to control movement of the object using the framework and to record a trajectory corresponding to movement of the object across a subject; the framework having a force applicator for applying a force to the subject in the z direction using the object and the control board being configured to record displacement in the x, y and z direction as the object moves along the trajectory; and the control board being configured to determine a stiffness map corresponding to the trajectory using displacement as a function of force.

In various embodiments, there may be included any one or more of the following features: the trajectory comprises a path of a light beam across the subject; the framework comprises x, y and z motors for moving the object; the trajectory is determined by sensors following movement of a device; the object comprises a roller; the stiffness map is associated with a pain record in a memory.

These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is a perspective view of an embodiment of a device to measure spinal stiffness.

FIG. 2A, 2B, 2C and 2D are respectively, afront view, side view, perspective view and bottom view of an embodiment of a roller used in the device of FIG. 1.

FIG. 3 is an example schematic of several components of a spinal assessment device.

FIG. 4 is a diagram of an example procedure for spinal assessment.

FIGS. 5A-C are screenshots of data collected from a single trial of a device with a lON vertical load. FIG. 5A shows head to toe movement of the gantry, FIG. 5B shows side to side movement of the gantry and FIG. 5C shows vertical displacement of the rod during the head to toe movement.

FIG. 6A shows an example curvilinear trajectory. FIG. 6B shows an example display for the trajectory in FIG. 6A with multiple masses.

FIG. 7A shows an example multi-direction trajectory. FIG. 7B shows an example display for the trajectory in FIG. 7A with multiple masses.

### DETAILED DESCRIPTION

Immaterial modifications may be made to the embodiments described here without departing from what is covered by the claims.

The probe is based on indentation where a stepping motor is used to press into the skin overlying a vertebra in a subject who is lying on their stomach. This probe must then be moved to a new vertebra where the vertebrae must first be found, then device aligned and then the process repeated. The indentation force and the resulting displacement of indentation probe are recorded. Stiffness is then calculated by determining the applied force divided by the resulting displacement.

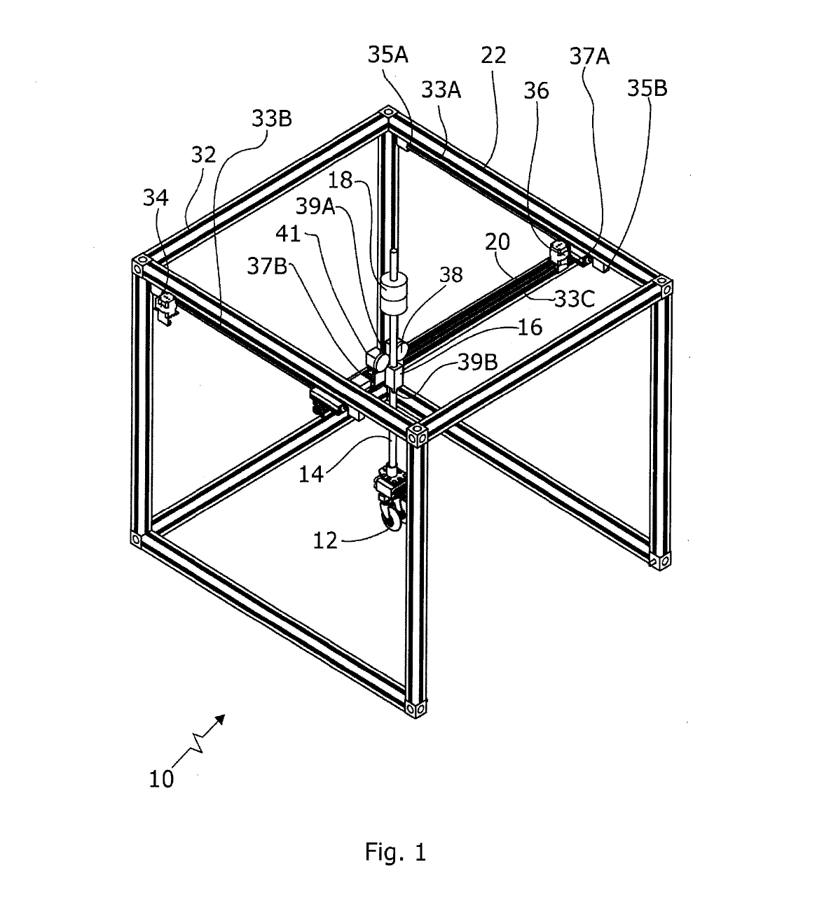

Referring to FIG. 1, the device 10 consists of a framework 22 supporting a roller 12 that is rolled over the surface of the skin, typically the surface of the back. A person being evaluated with the device 10 may lie on a table, bed, stretcher or bench that is placed under the roller 12 and within the framework 22. One end of the framework 22 may be open to allow the patient to easily enter within the framework 22. In an embodiment, the roller is a wheel which is mounted on the end of a rod 14 which is constrained within the Z-axis by a linear bearing 16 yet free to move in the vertical direction. A platform 18 on the rod allows increasing mass to be placed on the rod 14 and therefore allows the wheel to push down with more (or less) force as desired. The mass can be applied in physical increments mounted to the mass platform or through a motor designed to provide a continuous force magnitude. The rod with its wheel and mass platform are supported by a gantry 20 that can be moved in all horizontal directions (X-axis and Y-axis). The gantry is supported by a frame 32 that can be rolled over a treatment table. This allows the device to be used on subjects that are lying prone but other orientations of the device and subject are possible to assess the body in different circumstances (e.g. weight bearing, prone, static postures). The gantry 20 can be moved within the horizontal plane (X and Y axes) by hand or motor. The rod 14 with its attachments can be raised and lowered in the vertical plane either by hand or through an attached motor.

In FIG. 1, an x axis motor 34 may move the gantry 20 in the x axis. The gantry 20 is provided with limit switches 35A and 35B in the x direction. In FIG. 1, a y axis motor 36 may move the rod 14 along the gantry 20 in the y axis. The gantry 20 is provided with limit switches 37A and 37B in the y direction. The motors 34 and 36 may be stepper motors. Since the motors 34 and 36 are stepper motors then a stepper motor controller used to control the motors 34 and 36 may act as a sensor by keeping track of the steps to sense location of the respective motors 34, 36 hence the gantry 20 and rod 14. The motors 34 and 36 may be integrated with a belt drive system. Two belts 33A and 33B may be used in parallel on the x axis at either side of the gantry 20. The motor 34 drives one of the belts 33A and 33B, while the gantry 20 provides a connection so that driving of one of the belts drives the other. The motor 36 drives a single belt 33C on the gantry 20 and the linear bearing 16 that supports the rod 14 is moved by the belt 33C. Limit switches on each axis prevent excess movement in any direction if moved by motor. In all planes, sensors record displacement of the gantry and rod continuously to a level of accuracy not obtained with tools needing to be read by eyesight. These sensors could be integrated within the motor responsible for moving a particular axis or used independent of the motor. A z axis motor 38 with limit switches 39A and 39B is also provided on the gantry 20 to move the rod 14 up and down. The Z axis motor 38 does not need to be a stepper motor. Motor 38 is turned off during the evaluation phase and the rod moves freely in the z direction. To record the position of the rod when the motor is off, a z axis displacement sensor 41 is used to record movement of the rod 14 in the z direction.

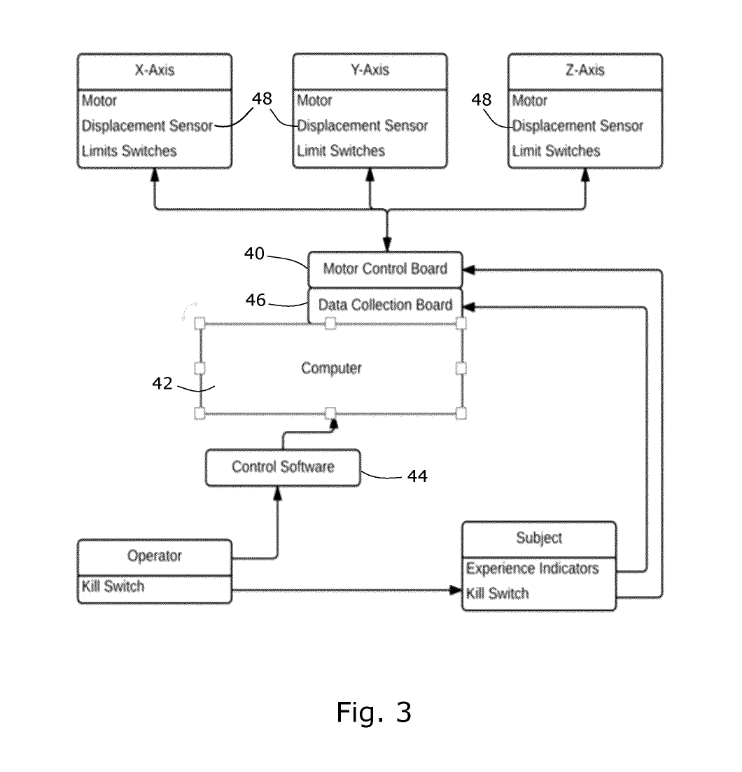

In an embodiment that uses motors to control movement in all axes, a motor control board 40 acts to control the direction, speed and position of the motors 34, 36 and 38. There may be a separate motor for each axis. The motor control board 40 is connected to a computer 42 which an operator uses to interface with customized software 44 that sends motor control parameters, as shown in an example schematic in FIG. 3. Similarly, a data collection board 46 attached to the computer collects sensor data related to the position of the device in all axes. The data collection board 46 also obtains information about the subject's experience (e.g. discomfort level) through various electronic indicators controlled by the subject.

The control board may comprise one or more electronic elements including a motor control board 40, data collection board 46, and control software residing in a computer. The motors may comprise linear actuators. The sensors may be displacement sensors. Any of various commercially available motors, data boards, memory devices, controllers, computers and sensors may be used. Individual functions may be carried out in individual devices or be spread across devices and elements of the control systems and other computing devices may reside externally to the system and be connected to the system by wired or wireless networks.

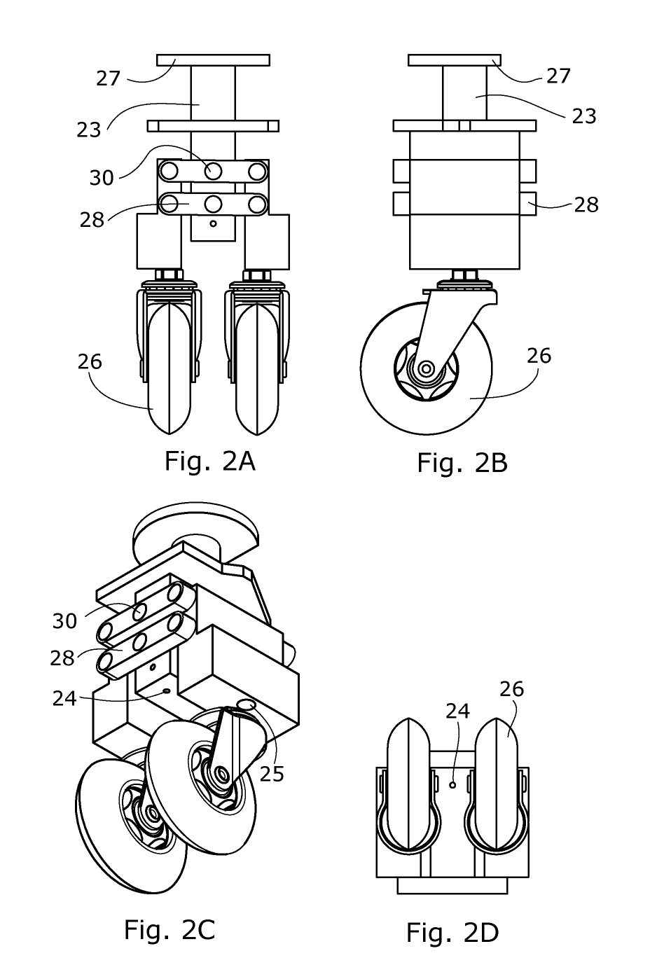

The roller may comprise two wheels 26 aligned to be parallel to one another, as shown in FIGS. 2A-2D, that are secured to the rod 14 for example with a rod receiver 23 and flange 27. The wheels may move in the vertical allowing the applied load to be better distributed from left to right. This helps when the spine is not equal in its topology to prevent one wheel from putting all the load on one side of the spine. The wheels may move independently or be coupled so a displacement of one wheel in one direction corresponds to a displacement of the other wheel in the opposite direction. The wheels may be coupled through a pivot 28 with a pivot axis 30. The pivot axis 30 may be centered between the two wheels. The wheels may swivel which allows the wheels to align to a change in left/right direction rather than the current situation where a left/right change in direction simply drags the static, forward facing wheels from side to side. The swiveling wheels may be positioned substantially behind a central axis of the rod when in operation.

The roller may be provided with the ability to change the distance between the two wheels 26 to accommodate different spine sizes. In an embodiment, this is achieved by a series of pre-drilled holes 25 and securing for example by screwing the wheels 26 into the desired holes, though a variety of mechanisms could be used to provide adjustable side to side positioning of the wheels 26. This also allows different wheels of different diameters to be swapped into the system should this be desired to accommodate subject size or dimensions.

In an example operation of the device a trajectory is obtained first. The object is then moved through the trajectory to develop the stiffness data. Also, there is a z motor which raises and lowers the object on/off the subject at the beginning/end of the test. Once the object is on the subject, the z motor is turned off so the object is free to move in the z direction with the displacement of all x, y, and z axes being obtained by the respective x, y and z sensors. A stiffness map corresponding to the trajectory is created using displacement as a function of force.

With the use of electronic sensors 48 to determine the displacement of each axis, it is possible to record the positions of these axes at any time by having the operator activate a command on the software and/or a hand switch that triggers the same operation. As a result, it is possible to move the rod to specific locations by motor or hand and then have the data collection board 46 record these axes position. The result is a series of data coordinate points that when connected to each other, create trajectory for the stiffness test. Similarly, a series of points can also be created by moving the rod over top of specific locations on the subject that relate to anatomic landmarks or predetermined positions identified by the operator or clinician. To ensure that the rod aligns with the desired points, a laser or light 24 mounted on the terminal end of the rod can then be used to accurately align the rod with the positions identified on the subject. By collecting axes position data at each point where the laser 24 aligns with the desired location of the subject, a specific trajectory can be created that can measure tissue stiffness along a desired pathway (i.e. trajectory) on the subject. The trajectory is calculated by the motor control board from this series of collected points.

The laser or light 24 may be embedded into the roller 12 or may be removably mounted on the terminal end of the rod. The laser or light may be positioned so the laser or light beam passes through an imaginary line connecting the axles of each wheel but if not, mathematical accommodation can be made to the trajectory as long as the laser position is known with respect to the central axis of the rod.

With the trajectory calculated, the rod is returned to a home position before being lowered until the roller 12 is in contact with the subject. At the operator's command, the wheel at the terminus of the rod can then be rolled through the path designated by the trajectory with the rod free to move in the vertical axis. The resulting displacement of the rod as it moves through this trajectory is a function of the applied mass and the displacement response of the subject. Additional mass can then be applied by the operator and the process repeated. Mass may be applied by adding increments of physical weight by hand. This process could be adapted to have the weights move on/off the rod via an automated, mechanical system or have an additional motor supply a continuous load to the rod. In this way, a continuous measure of stiffness is created in relation to its current location in the X and Y axes. Alternatively the rod may not be returned to home position but instead travel the trajectory in the reverse direction. The wheels may be swiveled 180 degrees to allow the rod to travel in the reverse direction.

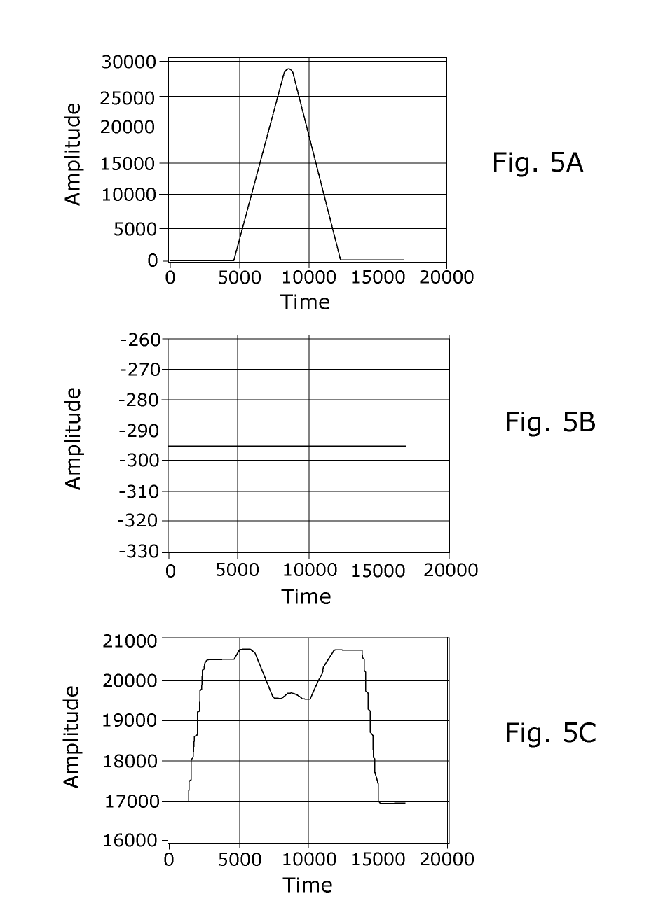

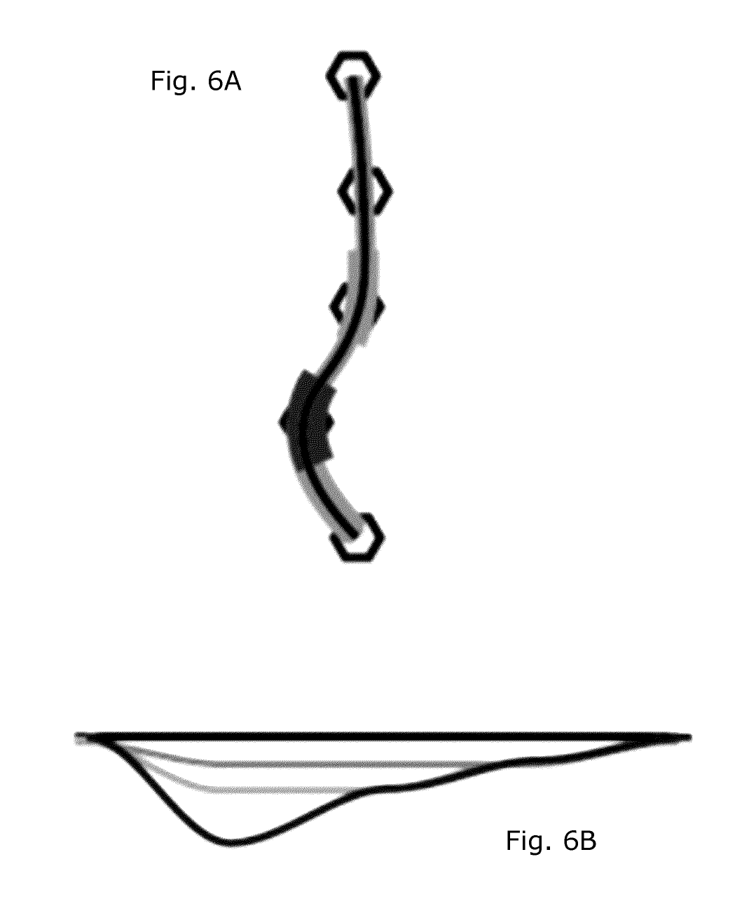

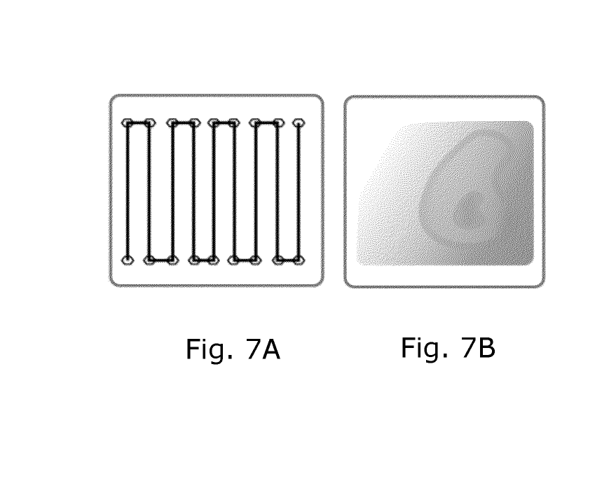

As a result of using the device, a three-dimensional assessment of spinal stiffness is created. From this, data stiffness is measured in a continuous fashion by taking the applied mass and dividing it by the instantaneous vertical displacement of the wheel/rod/mass platform. This information is then visualized within the two dimensional pathway that the wheel/rod/mass platform is moved in the horizontal plane for the desired trajectory. The process is then repeated with additional mass in an iterative fashion. As a result, a three-dimensional measure of stiffness is developed for the given trajectory over a series of applied masses. Depending on the trajectory, this stiffness information may pertain to a region of the back, the vertebrae specifically or the non-vertebral soft-tissues specifically. Example data for heat to toe and side to side movement of the gantry and vertical displacement of the rod during head to toe movement from a single trial with a 10N vertical load is shown in FIGS. 5A-C. If a single trajectory is used, data may be displayed to show the three dimensional nature of the trajectory and the resulting displacements, for example as shown in FIGS. 6A and 6B. Similarly, multidirectional trajectories can be used and the resulting data displayed in gradients of stiffness that can be portrayed as topographical regions related to stiffness, for example as shown in FIGS. 7A and 7B.

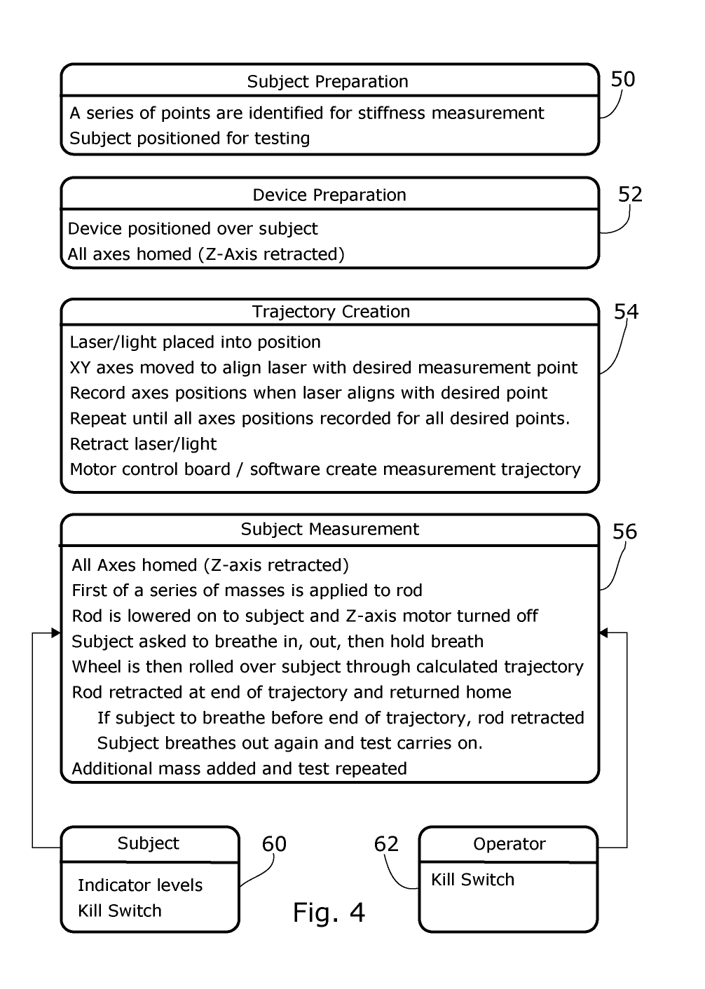

An example method of using the device is shown in FIG. 4. In a use of the device, a subject 60 having an exposed back is asked to lie on their stomach (50) on a rigid treatment table. The device is then positioned over the subject (52) and the rod retracted so that it does not touch the subject. A laser or light attached to the rod is swung into position to be in-series with the rod so it shines directly down on the subject in relation to the current rod position. With the horizontal motors deactivated, the operator moves the gantry over the desired trajectory points to be measured. Alternatively, the motors can be controlled by the operator to arrive at these same locations. At each point on the subject that defines the desired trajectory to be assessed, the operator moves the motors in the horizontal plane until the laser/light is aligned with the desired trajectory point. The operator 62 then activates the software to record the motor coordinates at this position. This process is repeated until the all points along the desired trajectory (straight or curvilinear) or region (rows and columns) are recorded into the system in a contiguous fashion (54). Using the motor control software, the wheel/rod/mass platform is lowered on to the subject beginning with no additional mass. Instructing the subject to breathe in, then out, then hold their breath at full expiration, the operator uses the software to instruct the device to lower the rod onto the subject's back then move the wheel through the desired trajectory with the rod free to move vertically as it follows the contours of the spine. When the trajectory is completed, the wheel is lifted off the subject by the software and the horizontal motors return the rod to the starting point of the trajectory. If the subject needs to breathe before the trajectory is complete, the wheel is raised, the patient allowed to collect their breath, then the process continued. The process is then repeated with additional mass (56). The system has emergency shutdown controlled by the operator and the subject. In addition, the subject controls one or a series of indictors that send a signal to the data collection system. These signals can be used to supply a continuous measure related to the subject's experience (e.g. discomfort). This signal is synced with the other data show that the level indicated by the subject can be collected as a continuous variable in relation to the position data. In this way, the system records a 4th dimension to the data by superimposing a subject indication (e.g. pain level) over the three-dimensional stiffness data.

To accurately determine the stiffness of the vertebrae, disc, facets, and other spinal components, an apparatus should be able to follow the minor or major deviations in spine alignment. The device, or Vertetrack, allows the user to derive true spinal stiffness by being able to assess each vertebra no matter how they are aligned. This is done by the operator creating a custom trajectory to assess specific the stiffness of the back in specific locations. With a laser placed in series with the rod, the operator simply moves the device to the points along the spine that Vertetrack should trace. If desired, these points can be identified and marked in advance. When the laser aligns with a desired point where spine stiffness should be quantified, the coordinates of the device at that position are recorded by the system. As many or as few points as needed can be collected. The result is a series of coordinates that define the position of each vertebrae and as a result, the trajectory to be followed by Vertetrack so that an accurate representation of spinal stiffness can be generated. This trajectory is created by a special controller that calculates the specific curvilinear trajectory that allows the roller to pass through each of the desired points with constant velocity no matter the vertebra's location in the spine itself. The result is a geographically correct measurement of spinal stiffness at a constant velocity. As a result, clinicians are provided with an accurate measure of spine stiffness rather than a measure which comes from non-spinal tissues.

The system may be used to measure stiffness of paraspinal or other tissues. Although the spine is made up of vertebra whose stiffness is of interest to clinicians, the spine is also controlled by muscles that extend outward on each side of the spine. As much as clinicians desire stiffness measures obtained from the spine, they also desire measures of stiffness of the muscles associated with the spine. The same process of teaching the device a series of points to assess vertebral stiffness can also be used to specifically measure the stiffness of paravertebral tissues. In this way, clinicians can know which measures of stiffness pertain to which tissues. Taking this to its logical conclusion, the technology can not only map specific types of tissues by assessing specific spinal areas, it can map the entire back in a series of rows and columns which creates a comprehensive picture of back stiffness.

The device can produce data that is three dimensional in nature. As the ability to see the heart or other tissues in three dimensions improves understanding of the heart function, seeing spine stiffness in three dimensions allows the clinician to see not only if there are areas of increased or decreased stiffness, but where exactly in the spine these areas are located.

In addition to stiffness data collected, the subject may be given an interactive sensor to record feedback (e.g. pain levels). In this way, the three-dimensional data provided by Vertetrackhas an additional fourth dimension. With this subject-based information, the clinician cannot only see where the spine is excessively stiff or complaint, but which areas of the three-dimensional stiffness data is related to subjective input such as pain or changes in the subject's pain with increasing mass.

The device can compensate for subject breathing. We have shown in the past that if a subject is actively breathing during stiffness tests, the results are inaccurate. In the case of Vertetrack, a motor may control the vertical movement of the roller. When a subject needs to breath, the motor lifts the roller off the subject and stops the measurement. When the patient exhales, the wheel is lowered and the measurement continues. In this way, a measurement of stiffness is created that is completely free from breathing artifacts.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

1. A method comprising:
creating and recording a trajectory across a subject;
using a control board and an object supported by a framework and while applying a force along a direction z to the subject using the object, moving the object along the trajectory and recording displacement of the object in the direction z; and
using displacement as a function of force to determine a stiffness map corresponding to the trajectory.

2. The method of claim 1 in which creating a trajectory comprises moving a light beam across the subject and recording x and y coordinates of the light beam.

3. The method of claim 1 in which moving the object comprises operating motors on the framework.

4. The method of claim 1 in which creating a trajectory comprises moving the object and recording movement of the object using sensors.

5. The method of claim 1 in which the object comprises a roller.

6. The method of claim 1 in which recording displacement is carried out iteratively for different forces.

7. The method of claim 1 in which the trajectory is across a non-spine portion of a back of the subject.

8. The method of claim 1 in which the trajectory is across a spine of the subject.

9. The method of claim 1 in which the stiffness map is associated with a pain record.

10. The method of claim 1 in which the method is carried out only while the subject is holding breathing.

11. An apparatus comprising:
a control board;
a framework providing controlled movement of an object in x, y and z directions, the control board being connected to control movement of the object using the framework and to record a trajectory corresponding to movement of the object across a subject;
the framework having a force applicator for applying a force to the subject in the z direction using the object and the control board being configured to record displacement in the x, y and z direction as the object moves along the trajectory; and
the control board being configured to determine a stiffness map corresponding to the trajectory using displacement as a function of force.

12. The apparatus of claim 11 in which the trajectory comprises a path of a light beam across the subject.

13. The apparatus of claim 11 in which the framework comprises x, y and z motors for moving the object.

14. The apparatus of claim 11 in which the trajectory is determined by sensors following movement of a device.

15. The apparatus of claim 11 in which the object comprises a roller.

16. The apparatus of claim 11 in which the stiffness map is associated with a pain record in a memory.
